(19) United States
(12) Patent Application Publication
(10) Pub. No.: US 2007/0122480 A1
(43) Pub. Date: May 31, 2007

(54) Sustained release formulations

(75) Inventors: Seong Hwan Cho, Suwon-city (KR); Jeong Ku, Yongin-city (KR); Dong Kwon Lim, Yongin-city (KR); Jun Hee Cheon, Suwon-city (KR); Tae Kun An, Yongin-city (KR); Jae Kyoung Ko, Incheon-city (KR); Yong Sik Youn, Yongin-city (KR); Choong Sil Park, Icheon-city (KR); Hea Ran Suh, Icheon-city (KR); Eun Young Yang, Suwon-city (KR); Eun Kyung Jeon, Yongin-city (KR); Chang Ju Kim, Suwon-city (KR)

Correspondence Address: ROTHWELL, FIGG, ERNST & MANBECK, P.C., 1425 K STREET, N.W., SUITE 800, WASHINGTON, DC, 20005, US

(73) Assignee: CJ CORPORATION, Seoul (KR)

(21) Appl. No.: 10/574,337

(22) PCT Filed: Sep. 25, 2004
(86) PCT No.: PCT/KR04/02496

§ 371 Date: May 9, 2006

(30) Foreign Application Priority Data
Sep. 29, 2003 (KR) 10-2003-0067588
Sep. 24, 2004 (KR) 10-2004-0077158

(51) Int. Cl. A61K 9/24

(52) U.S. Cl. 424/472

(57) ABSTRACT

Provided is a sustained-release formulation: (a) a sustained-release core including an active ingredient and a polymer having erosion and swelling property in mammalian intestinal secretions, (b) an enteric film coating layer coated on the sustained-release core, and (c) an active ingredient-containing film coating layer coated on the enteric film coating layer and including the active ingredient and hydrophilic polymer for film coating.

### TECHNICAL FIELD

The present invention relates to a sustained-release formulation that provides a two-step release of an active ingredient in the gastrointestinal tract over a prolonged period of time when orally administered.

### BACKGROUND ART

Cardiovascular side effects such as orthostatic disorder are often observed in use of cardiovascular agents such as beta (β)-adrenergic receptor blocking agents and antidysrhythmic agents. Such cardiovascular side effects are mainly caused by rapid elevation of plasma drug concentration within a short time after administered. In this respect, sustained-release formulations that can lead to the controlled release of a drug are required.

A conventional sustained-release matrix formulation using a hydrophilic polymer has an advantage of simple and easy preparation. However, there is a disadvantage in that the formulation absorbs water in the digestive tract immediately after administered, thereby leading to an initial excessive release of a drug. Furthermore, even though an initial release of a drug is dependent on the concentration gradient of the drug, as dissolution of the drug proceeds, the release rate of the drug decreases due to a decrease of the concentration gradient and an increase of diffusion distance. For this reason, the sustained-release matrix formulation using the hydrophilic polymer is difficult to accomplish zero (0)-order release of a drug. In particular, it can be said that the sustained-release matrix formulation is not suitable for an orthostatic disorder-producing drug such as a cardiovascular agent including a β-adrenergic receptor blocking agent and an antidysrhythmic agent.

U.S. Pat. No. 4,252,786 discloses a controlled release formulation including a swellable matrix coated with hydrophobic and hydrophilic polymers, which can provide initial sustained release and zero-order release rate of a drug.

This formulation can retard an initial drug release by the coating during gelation of the drug-containing matrix. However, when a coating layer is broken, there arises a problem in that the release of a drug is dependent on the concentration gradient of the drug, like a common matrix formulation.

U.S. Pat. No. 5,464,633 discloses a technique that prevents an initial burst release of a drug by externally applying a compressed tablet layer, instead of a coating layer, to a matrix with swelling and erosion property. However, this technique involves a very complicated production process

Korean Patent Laid-Open Publication No. 1998-85592 discloses a sustained-release formulation including a drug-containing core and a double coating layer (i.e., double layer system) made of two or more polymeric materials. The formulation has been designed in such a way that swelling of a primary coating layer is controlled by a secondary coating layer. However, the preparation of the formulation is complicated.

An osmotic pump tablet including a core surrounded by a water-insoluble solid membrane such as a cellulose acetate membrane can provide a zero-order drug release. However, use of an organic solvent is required for film coating and laser drilling for hole formation in the osmotic pump tablet increases a process burden (US 1999-1713).

Therefore, a sustained-release formulation that can accomplish a near zero-order release by efficiently controlling an initial burst drug release is required.

### BRIEF DESCRIPTION OF THE DRAWINGS

FIG. 1 is a diagram that illustrates a sustained-release formulation according to an embodiment of the present invention.

FIG. 2 illustrates the results of dissolution tests for sustained-release formulations prepared in Examples 3, 4, and 5 according to the present invention.

FIG. 3 illustrates the results of dissolution tests for sustained-release formulations prepared in Examples 5, 6, and 7 according to the present invention.

FIG. 4 illustrates the result of dissolution test for a sustained-release formulation prepared in Example 8 according to the present invention.

### DETAILED DESCRIPTION OF THE INVENTION

Technical Goal of the Invention

The present invention provides a sustained-release formulation that can control a drug release according to a near zero-order release rate without an initial burst drug release.

Disclosure of the Invention

According to an aspect of the present invention, there is provided a sustained-release formulation including:

(a) a sustained-release core including an active ingredient and a polymer having erosion and swelling property in mammalian intestinal secretions;

(b) an enteric film coating layer coated on the sustained-release core; and

(c) an active ingredient-containing film coating layer coated on the enteric film coating layer and including the active ingredient and a hydrophilic polymer for film coating.

The sustained-release formulation may further include an outer coating layer coated on the active ingredient-containing film coating layer and including a polymer selected from the group consisting of a hydrophilic polymer, a hydrophobic polymer, a pH-dependent polymer, and a combination thereof.

The polymer contained in the sustained-release core and having erosion and swelling property in mammalian intestinal secretions may be a polymer having a viscosity of 1 to 100,000 mPa·s and preferably 3,500 to 100,000 mPa·s. The polymer may be selected from the group consisting of hydroxypropylmethylcellulose, hydroxypropylcellulose, hydroxyethylcellulose, hydroxymethylcellulose, polyethylene oxide, waxes (Carnauba wax), sodium alginate, povidone, polyvinylalcohol, sodium carboxymethylcellulose, xanthan gum, alginic acid salt and its derivative, and a combination thereof, but is not limited thereto. Most preferably, hydroxypropylmethylcellulose can be used.

The content of the polymer in the sustained-release core may be about 1 to 99 wt %, based on the total weight of the sustained-release core.

Meanwhile, the hydrophilic polymer contained in the active ingredient-containing film coating layer may be a hydrophilic polymer having a viscosity of 1 to 100,000 mPa·s and preferably 3,500 to 100,000 mPa·s. The hydrophilic polymer may be selected from the group consisting of polyvinylalcohol, polyethyleneglycol, polypropyleneglycol, acrylic acid copolymer, hydroxypropylmethylcellulose, hydroxypropylcellulose, methylcellulose, ethylcellulose, and a combination thereof, but is not limited thereto.

The enteric film coating layer coated on the sustained-release core may include an enteric polymer which is soluble at about pH 5 or more. The polymer contained in the outer coating layer may also be an enteric polymer which is soluble at about pH 5 or more.

The enteric polymer used for the enteric film coating layer and the outer coating layer may be selected from the group consisting of cellulosic polymers, polyvinyl polymers, maleic acid vinyl polymers, polymethacrylate copolymers, and combinations thereof, but is not limited thereto. Preferably, the enteric copolymer may be a 1:1 copolymer of methacrylic acid and ethylacrylate.

The active ingredient contained in the sustained-release formulation of the present invention may be selected from the group consisting of antihypertensive agents, antidiabetes agents, antilipemic agents, cardiovascular drugs, expectrorants, antibiotics, emollients, steroids, antiasthmatic drugs, nonsteroid anti-inflammatory agents, therapeutic agents for prostatic enlargement, antidepressants, antihistamines, and combinations thereof, but is not limited thereto.

The active ingredient may be nifedipine, felodipine, cetirizine, pseudoephedrine, tamsulosin, or a pharmaceutically acceptable salt thereof. In particular, tamsulosin or its hydrochloride is preferable.

When the active ingredient contained in the sustained-release formulation is tamsulosin or its hydrochloride, it is preferred that 60 to 99 wt % of tamsulosin is contained in the sustained-release core and 1 to 40 wt % of tamsulosin is contained in the active ingredient-containing film coating layer.

Hereinafter, the present invention will be described in more detail.

A sustained-release formulation according to the present invention can control a drug release according to a near zero-order release rate without an initial burst drug release. For this, the sustained-release formation includes separate two layers each containing a drug, an enteric film coating layer interposed between the two layers, and an optional outer coating layer controlling an initial drug release.

Therefore, the sustained-release formulation according to the present invention includes:

(a) a sustained-release core including an active ingredient and a polymer having erosion and swelling property in mammalian intestinal secretions;

(b) an enteric film coating layer coated on the sustained-release core; and

(c) an active ingredient-containing film coating layer coated on the enteric film coating layer and including the active ingredient and a hydrophilic polymer for film coating.

As used herein, the term “mammalian intestinal secretions” is used as the meaning including fluids present in the duodenum, the small intestine, and the large intestine among mammalian digestive tracts, and in particular, refers to fluids present in the small and large intestines.

The sustained-release core contains 5 to 99 wt % of the active ingredient in the sustained-release formulation.

The polymer contained in the sustained-release core and having erosion and swelling property in mammalian intestinal secretions may be a polymer having such a property which is commonly known in the pharmaceutics field. Preferably, the polymer contained in the sustained-release core is a polymer that can release the active ingredient gradually, preferably according to a zero-order release rate. The viscosity of the polymer contained in the sustained-release core is 1 to 100,000 mPa·s, preferably 3,500 to 100,000 mPa·s, and more preferably 4,000 to 20,000 mPa·s.

The polymer having the above-described viscosity property and erosion and swelling property may be hydroxypropylmethylcellulose, hydroxypropylcellulose, hydroxyethylcellulose, hydroxymethylcellulose, polyethylene oxide, waxes (Carnauba wax), sodium alginate, povidone, polyvinylalcohol, sodium carboxymethylcellulose, xanthan gum, alginic acid salt or its derivative, or a combination of one or more of the forgoing polymers.

Among the forgoing polymers, hydroxypropylmethylcellulose is most preferable. A commercially available Methocel K4M CR Premium (Dow Chemical, America) may be used.

The content of the polymer in the sustained-release core may vary according to the type of the polymer. However, the content of the polymer in the sustained-release core may be 1 to 99 wt %, and preferably 1 to 50 wt %, based on the total weight of the sustained-release core.

The sustained-release core may include common additives, in addition to the active ingredient and the polymer. For example, the sustained-release core may include a diluent such as a noncrystalline cellulose (e.g. Avicel), dextrose, starch, sucrose, lactose, sorbitol, mannitol, and calcium phosphate (dicalcium or tricalcium); a disintegrating agent such as talc or corn starch; a binder such as polyvinylpyrrolidone, magnesium aluminum silicate, starch paste, gelatin, tragacanth, methylcellulose, sodium carboxymethylcellulose, hydroxypropylcellulose, and copovidone; and a solvent such as water or a lower alcohol such as ethanol and isopropanol; and a lubricant such as light anhydrous silicic acid, talc, stearic acid and its zinc, magnesium, or calcium salt, and polyethyleneglycol. In addition, the sustained-release core may include a disintegrating agent such as sodium starch glycolate (e.g. Primojel), starch, alginic acid or its sodium salt, an azeotropic mixture, an absorbant, a colorant, a flavoring agent, or a sweetener.

When the noncrystalline cellulose is used as a diluent, the noncrystalline cellulose may be used in an amount of about 10 to 90 wt %, and preferably about 60 to 90 wt %, based on the total weight of the sustained-release core.

The lubricant may be used in an amount of 0.2 to 2 wt %, and preferably about 1 wt %, based on the total weight of the sustained-release core. The disintegrating agent may be used in an amount of 0.2 to 5 wt %, and preferably about 2 wt %, based on the total weight of the sustained-release core. Polyvinylpyrrolidone used as a binder when needed may be used in an amount of 1 to 20 wt %, and preferably about 2 to 10 wt %, based on the total weight of the sustained-release core.

The sustained-release formulation of the present invention includes the enteric film coating layer coated on the sustained-release core. The enteric film coating layer serves to prevent the release of the active ingredient contained in the sustained-release core and continuously release only the active ingredient contained in the active ingredient-containing film coating layer without initial excessive release of the active ingredient in the gastric tract.

A polymer constituting the enteric film coating layer may be a commonly used enteric polymer. Preferred is an enteric polymer that is soluble at about pH 5 or more. The enteric polymer as used herein may be selected from the group consisting of cellulosic polymers, polyvinyl polymers, maleic acid vinyl polymers, polymethacrylate copolymers, and combinations thereof.

Examples of the cellulosic polymers include cellulose acetate phthalate, cellulose acetate succinate, methylcellulose phthalate, hydroxymethylethylcellulose phthalate, hydroxypropylmethylcellulose phthalate, and hydroxypropylmethylcellulose acetate succinate. Examples of the polyvinyl polymers include polyvinylalcohol phthalate, polyvinylbutyrate phthalate, and polyvinylacetoacetyl phthalate. Examples of the maleic acid vinyl polymers include poly(vinylacetate, maleic anhydride), poly(vinylbutylether, maleic anhydride), and poly(styrene, maleic monoester). Examples of the polymethacrylate copolymers include polymethacrylate, poly(methacrylate-ethylacrylate), methylmethacrylate, and (one or two triethylaminoethyl) copolymers.

More preferably, the enteric polymer that is soluble at pH 5 or more may be selected from the group consisting of cellulose acetate phthalate, polyvinylacetate phthalate, hydroxypropylmethylcellulose phthalate, polymethacrylate copolymer, and a combination thereof. A 1:1 copolymer of methacrylic acid and ethylacrylate (poly(methacrylic acid, ethyl acrylate) 1:1) is still more preferable. As the 1:1 copolymer of methacrylic acid and ethylacrylate, there may be used Eudragit L100, L12.5, L12.5P, 30D-55, and L100-55, which are commercially available. Eudragit L100-55 is preferable.

The enteric film coating layer may include a commonly used plasticizer, in addition to the enteric polymer. Examples of the plasticizer include polyethyleneglycol and its derivative (e.g.: PEG 6000), a fatty acid, a substituted triglyceride, glyceride, castor oil, and hydrogenated castor oil. The plasticizer may be contained in a coating solution for the enteric film coating layer in an amount of 5 to 50%. The coating solution may also include 0.1 to 2 wt % of sodium hydrogen carbonate and/or sodium lauryl sulfate when needed.

The enteric film coating layer may constitute about 1 to 20 wt % of the sustained-release formulation, and preferably about 8 to 12 wt %.

Meanwhile, as described above, the sustained-release formulation of the present invention includes the active ingredient-containing film coating layer coated on the enteric film coating layer and including a portion of the active ingredient and a hydrophilic polymer for film coating.

The hydrophilic polymer used for the active ingredient-containing film coating layer may be a common hydrophilic polymer that can accomplish film coating. The content of the hydrophilic polymer is preferably maintained at a minimal level to limit the size of the formulation and ensure an efficient preparation. The content of the hydrophilic polymer may be about 1 to 10 wt %, and preferably about 3 to 8 wt %, based on the total weight of the sustained-release formulation.

The hydrophilic polymer used for the active ingredient-containing film coating layer may have a viscosity of 1 to 100,000 mPa·s, preferably 3,500 to 100,000 mPa·s, and more preferably 4,000 to 20,000 mPa·s.

Preferably, the hydrophilic polymer having the above-described viscosity property may be acrylic acid copolymer, polyoxyethylene sorbitan ester, or a cellulose compound such as hydroxypropylmethylcellulose, hydroxypropylcellulose, methylcellulose, and ethylcellulose. The content of the hydrophilic polymer in the active ingredient-containing film coating layer is preferably maintained at a minimal level to limit the size of a final formulation, for example a tablet, and ensure an efficient preparation.

The active ingredient-containing film coating layer may further include polyethyleneglycol, for example PEG 6000, a plasticizer such as triacetin, and the like. The active ingredient-containing film coating layer may also include talc and titanium dioxide to facilitate coating.

The active ingredient of the sustained-release formulation of the present invention is appropriately distributed in the sustained-release core and the active ingredient-containing film coating layer. For example, when tamsulosin or its hydrochloride is used as the active ingredient, it is preferred that 60 to 90 wt % of the tamsulosin or its hydrochloride is contained in the sustained-release core and 10 to 40 wt % is contained in the active ingredient-containing film coating layer.

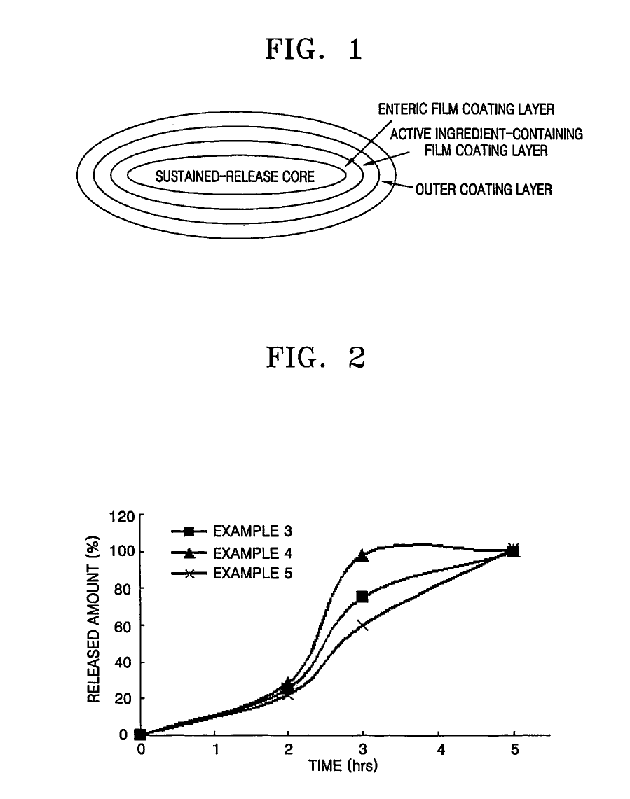

When needed, the sustained-release formulation of the present invention may further include an outer coating layer externally applied to the active ingredient-containing film coating layer so as to prevent the initial burst elevation of plasma active ingredient concentration in the body and to exhibit sustained-release characteristics in the early phase of administration. The outer coating layer also serves to continuously release only the active ingredient contained in the active ingredient-containing film coating layer in the early phase of administration and to impart wear resistance, stability, and functionality to the active ingredient. A diagram illustrating a sustained-release formulation including an outer coating layer according to an embodiment of the present invention is shown in FIG. 1.

The outer coating layer may include at least one a film coating polymer selected from the group consisting of hydrophilic, hydrophobic, and pH-dependent polymers. In particular, the outer coating layer may include a polymer selected from the group consisting of cellulosic polymers, polyvinyl polymers, maleic acid vinyl polymers, polymethacrylate copolymers, and combinations thereof. A 1:1 copolymer of methacrylic acid and ethylacrylate is still more preferable. As the 1:1 copolymer of methacrylic acid and ethylacrylate, there may be used Eudragit L100, 30D-55 and L100-55 which are commercially available. In particular, Eudragit L100-55 is preferable.

The outer coating layer may further include a commonly used plasticizer, for example, polyethyleneglycol or its derivative, a fatty acid, a substituted triglyceride, glyceride, castor oil, or hydrogenated castor oil. When needed, the outer coating layer may also include 0.1 to 2 wt % of sodium hydrogen carbonate and/or sodium lauryl sulfate.

The content and type of the polymer in the outer coating layer may vary according to a desired dissolution pattern. That is, as the content of the polymer increases, the release rate of the active ingredient contained in the formulation decreases, and in particular, an initial release rate of the active ingredient remarkably decreases. In this respect, the sustained-release formulation of the present invention can efficiently control an initial release rate of the active ingredient by adjusting the coating amount of the outer coating layer. The polymer content, i.e., coating amount, of the outer coating layer, even though varies according to a desired dissolution pattern, ranges from about 0.1 to 20 wt %, and preferably about 0.1 to 5 wt %, based on the total weight of the sustained-release core.

The coating may be carried out according to a common coating method. A water-based coating using a pan coating system for tablet coating is preferable. That is, it is preferable to perform the coating for the sustained-release formulation by spraying an aqueous suspension of the polymer. In this case, since the coating is performed using the aqueous suspension instead of an organic solvent, an environmental contamination problem by use of the organic solvent may not be caused.

A pharmaceutical composition of the present invention can be formulated as various types of oral formulations having the above-described composition. Preferably, the pharmaceutical composition of the present invention can be formulated in a tablet form. An illustrative preparation of a tablet form is as follows.

The sustained-release core contained in the sustained-release formulation of the present invention can be prepared by direct compression or compaction-granulation.

In the case of using direct compression, the sustained-release core can be prepared in such a manner that an active ingredient, a swellable polymer (e.g. Methocel K4M CR Premium), a direct compression diluent (e.g. Avicel PH102), and a disintegrating agent (e.g. Primojel) are mixed and then a lubricant such as magnesium stearate is further added thereto, followed by tabletting.

In the case of using compaction-granulation, the sustained-release core can be prepared in such a manner that an active ingredient, a swellable polymer (e.g. Methocel K4M CR Premium), a diluent (e.g. Avicel PH101), a disintegrating agent (e.g. L-HPC), a binder (e.g. HPC-L), and a lubricant (e.g. magnesium stearate) are mixed, followed by granulation with a compaction granulator (e.g. roller compacter), screening through a about 20-mesh screen, and tabletting.

The enteric film coating layer can be formed by a common coating process. For example, in the case of using water-based coating by a common pan coating system, the enteric film coating layer can be formed in such a manner that a suspension obtained by suspending Acryl-Eze (a coating system including Eudragit L100-55, commercially available from Colorcon) in water is screened to obtain a coating solution, a sustained-release core is placed in a pan coater (e.g. Hi-coater), coating is performed at an inlet air temperature of 50 to 80° C. and an outlet air temperature of about 30 to 45° C., followed by drying using a common method (e.g., drying with a dry air for 30 minutes).

The active ingredient-containing film coating layer can also be formed by a common coating method, like in the formation of the enteric film coating layer. For example, in the case of using water-based coating by a common pan coating system, the active ingredient-containing film coating layer can be formed in such a manner that Opadry (coating system including 45.52% of PVA, commercially available from Colorcon) is suspended in an active ingredient-containing solution obtained by dissolving an active ingredient in water to prepare a coating solution, an enteric film-coated sustained-release core is placed in a pan coater (e.g., Hi-coater), coating is performed at an inlet air temperature of 50 to 80° C. and an outlet air temperature of about 30 to 45° C., followed by drying using a common method (e.g., drying with a dry air for 30 minutes).

The outer coating layer can also be formed using the same coating method as in the formation of the enteric film coating layer.

The sustained-release formulation thus prepared may be a tablet. In this case, the sustained-release formulation can be stored in a water vapor barrier vessel such as a blister pak (Alu-Alu; PVDC, PE, PVC-Alu). The sustained-release formulation can also be formulated in a hard capsule form by filling it in a hard capsule.

The sustained-release formulation according to the present invention as prepared in the above can accomplish a two-step drug release. That is, a drug contained in the active ingredient-containing film coating layer is continuously released in a gastric fluid in the early phase of oral administration. After then, the enteric film coating layer is destroyed within several minutes in the small intestine and a drug contained in the sustained-release core is continuously released in the small intestine. Therefore, a drug release can be controlled according to a zero-order release rate without an initial burst drug release.

The sustained-release formulation according to the present invention can be applied to, as an active ingredient, a drug requiring the prevention of its burst plasma concentration elevation in the body in the early phase of administration and sustained-release characteristics. For example, the sustained-release formulation according to the present invention can be applied to antidiabetes agents (glymepiride, glipizide, gliclazide, mefformin, and a therapeutically equivalent salt thereof), antihypertensive agents (irvesartan, fosinopril, felodipine, lercanidipine, lasidipin, nicardipine, amosulalol, perindopril, clizapril, imidapril, lisinopril, losartan, doxazosin, candesartan), antilipemic agents (simvastatin, lovastatin), cardiovascular drugs, expectrorants, antibiotics, emollients, steroids, antiasthmatic drugs, nonsteroid anti-inflammatory agents, and the like. In particular, the sustained-release formulation according to the present invention can be more preferably applied to tamsulosin or its hydrochloride used as a therapeutic agent for prostatic enlargement.

Effect of the Invention

A sustained-release formulation according to the present invention can efficiently prevent a burst drug release in the early phase of oral administration and continuously release a drug during a prolonged period of time. Furthermore, since the coating for formation of an enteric film coating layer and an outer coating layer constituting the sustained-release formulation of the present invention can be carried out by a common coating process, a specific process apparatus is not required. In addition, since a water-based coating process can be used, an environmental contamination problem by use of an organic solvent can be prevented.

### BEST MODE FOR CARRYING OUT THE INVENTION

Hereinafter, the present invention will be described more specifically by Examples. However, the following Examples are provided only for illustrations and thus the present invention is not limited to or by them.

### EXAMPLE 1

(1) Preparation of Sustained-release Cores

Sustained-release cores were prepared according to the composition of an active ingredient and additives (weight: mg) as presented in Table 1 below.

TABLE 1

| Section | Weight (mg) |
|---|---|
| Felodipine | 4.17 |
| Hydroxypropylmethylcellulose | 70 |
| Avicel PH102 | 35.2 |
| Magnesium stearate | 2 |
| Sustained-release core | 111.42 |

According to the composition ratio as presented in Table 1, felodipine, hydroxypropylmethylcellulose (hipromelos 2903 mPa·s), and a direct compression diluent (Avicel PH102) were mixed in a mixer. Magnesium stearate was added thereto and completely mixed. The resultant mixture was compacted in a rotary press (Korsch PH 106) to make 100,000 white tablets (i.e. sustained-release cores, 111.42 mg for each).

(2) Enteric Film Coating

Eudragit L30 D-55 (30% aqueous suspension, 14.3 kg), PEG 6000 (10% aqueous solution, 4.15 kg), talc (1.1 kg), and cremophor EL (0.05 kg) were gradually added to water and stirred until completely dissolved to prepare an enteric film coating solution. The sustained-release cores prepared in Section (1) were placed in a coating pan (i.e., Glatt type GC-750) and warmed by air so that an outlet air temperature reached about 30 to 40° C. The enteric film coating solution was sprayed on the sustained-release cores by an air pressure-propelled sprayer. At the time of terminating the spraying, heating was stopped but air supply was continued for about 30 minutes to dry the tablets. The coated cores were isolated from the sprayer, dried with dry air, and left stand at room temperature for 24 hours.

(3) Preparation and Coating of Drug-containing Film Coating Suspension

Hydroxypropylmethylcellulose 2910 mPa·s (12.99 kg), talc (0.45 kg), and triacetin (1.5 kg) were gradually added to water (35.007 kg), which had been heated to about 70° C., with stirring, and then water (17.503 kg) was added thereto. Then, felodipine (0.85 kg) was gradually added to prepare a drug-containing film coating suspension. The drug-containing film coating suspension was homogenized and cooled to room temperature with continuously stirring. The enteric film-coated sustained-release cores prepared in Section (2) were placed in a coating pan and warmed by air so that an outlet air temperature reached about 40 to 50° C. The drug-containing film coating suspension was sprayed on the enteric film-coated sustained release cores. The resultant drug-coated tablets were dried by air supply for about 10 minutes, isolated from the coating pan, and stored.

The sustained-release tablets thus prepared had an average weight of 145.81 mg and each contained a 5 mg (total) of felodipine.

### EXAMPLE 2

(1) Preparation of Sustained-release Cores

In this Example, sustained-release cores had a composition ratio as presented in Table 2 below and a preparation thereof was as follows.

TABLE 2

| Section | Weight (mg) |
|---|---|
| Nifedipine | 27.5 |
| hydroxypropylmethylcellulose | 70 |
| Avicel PH102 | 35.2 |
| Magnesium stearate | 2 |
| Sustained-release core | 134.75 |

According to the composition ratio as presented in Table 2, nifedipine, hydroxypropylmethylcellulose (hipromelos 2903 mPa·s), and a direct compression diluent (Avicel PH102) were mixed in a mixer. Magnesium stearate was added thereto and completely mixed. The resultant mixture was compacted in a rotary press (Korsch PH 106) to make 100,000 white tablets (i.e. sustained-release cores, 134.75 mg for each).

(2) Enteric Film Coating

Eudragit L30 D-55 (30% aqueous suspension, 14.3 kg), PEG 6000 (10% aqueous solution, 4.15 kg), talc (1.1 kg), and cremophor EL (0.05 kg) were gradually added to water and stirred until completely dissolved to prepare an enteric film coating solution. The sustained-release cores prepared in Section (1) were placed in a coating pan (i.e., Glatt type GC-750) and warmed by air so that an outlet air temperature reached about 30 to 40° C.

The enteric film coating solution was sprayed on the sustained-release cores by an air pressure-propelled sprayer. At the time of terminating the spraying, heating was stopped but air supply was continued for about 30 minutes to dry the tablets. The coated cores were isolated from the sprayer, dried with dry air, and left stand at room temperature for 24 hours.

(3) Preparation and Coating of Drug-containing Film Coating Suspension

Hydroxypropylmethylcellulose 2910 mPa·s (12.99 kg), talc (0.45 kg), and triacetin (1.5 kg) were gradually added to water (35.007 kg), which had been heated to about 70° C., with stirring, and water (17.503 kg) was added thereto. Then, nifedipine (5.5 kg) was gradually added to prepare a drug-containing film coating suspension. The drug-containing film coating suspension was homogenized and cooled to room temperature with continuously stirring. The enteric film-coated sustained-release cores prepared in Section (2) were placed in a coating pan and warmed by air so that an outlet air temperature reached about 40 to 50° C. The drug-containing film coating suspension was sprayed on the enteric film-coated sustained-release cores. The resultant drug coated tablets were dried by air supply for about 10 minutes, isolated from the coating pan, and stored.

The sustained-release tablets thus prepared had an average weight of 174.75 mg and each contained a 33 mg (total) of nifedipine.

### EXAMPLES 3 THROUGH 5

(1) Preparation of Sustained-release Cores

In these Examples, sustained-release cores had composition ratios as presented in Table 3 below and a preparation method thereof was as follows.

TABLE 3

| | Weight (mg) | | |
|---|---|---|---|
| Section | Example 3 | Example 4 | Example 5 |
| Tamsulosin hydrochloride | 0.15 | 0.15 | 0.15 |
| Methocel K4M CR Premium | 50 | 20 | 70 |
| Avicel PH102 | 47.85 | 77.85 | 27.85 |
| Magnesium stearate | 1 | 1 | 1 |
| Primojel | 1 | 1 | 1 |
| Sustained-release core | 100 | 100 | 100 |

According to the composition ratios as presented in Table 3, tamsulosin hydrochloride (0.15 kg), Methocel K4M CR Premium (Dow Chemical, America), a direct compression diluent (Avicel PH102), and Primojel were mixed in a mixer. Magnesium stearate was added thereto and completely mixed. The resultant mixture was tabletted in a rotary press (Korsch PH 106) to make 100,000 white sustained-release cores (100 mg for each).

(2) Enteric Film Coating

5 kg of the sustained-release cores prepared in Section (1) were placed in a coating pan (Hi-coater) and warmed by air so that an outlet air temperature reached about 30 to 40° C. A coating solution prepared by suspending 500 g of Acryl-Eze (a 40% Eudragit L100-55-containing coating system, Colorcon) in 4-fold water was sprayed on the sustained-release cores by an air pressure-propelled sprayer and dried by air supply for about 10 minutes. The coating amount of Acryl-Eze (a 40% Eudragit L100-55-containing coating system, Colorcon) in the resultant tablets was 10% of the total weight of the sustained-release cores.

(3) Preparation and Coating of Drug-containing Film Coating Suspension

The coating pan (Hi-coater) containing the dried tablets prepared in Section (2) was maintained at an outlet air temperature of about 30 to 40° C. A coating solution prepared by suspending 2.5 g of tamsulosin hydrochloride and 240 g of Opadry (a 45.52% PVA-containing coating system, Colorcon) in 4-fold water was sprayed on the dried tablets by an air pressure-propelled sprayer and dried by further air supply for about 10 minutes. The coating amount of Opadry (a 45.52% PVA-containing coating system, Colorcon) in the resultant tablets was 4% of the total weight of the sustained-release cores.

(4) Preparation and Coating of Outer Film Coating Suspension

The coating pan (Hi-coater) containing the dried tablets prepared in Section (3) were maintained at an outlet air temperature of about 30 to 40° C. A coating solution prepared by suspending 12 g of Acryl-Eze (a 40% Eudragit L100-55-containing coating system, Colorcon) in 4-fold water was sprayed on the dried tablets by an air pressure-propelled sprayer and dried by further air supply for about 10 minutes. The coating amount of Acryl-Eze (a 40% Eudragit L100-55-containing coating system, Colorcon) in the resultant tablets was 2% of the total weight of the sustained-release cores.

The finally obtained tablets of Examples 3 through 5 had an average weight of 116 to 117 mg.

(5) Dissolution Test

The dissolution tests for the tablets prepared in Examples 3 through 5 were performed according to the second method of the dissolution test in the Korean pharmacopoeia. At this time, during initial two hours, the dissolution tests were performed in dissolution media prepared by accurately adding 1 ml of polysorbate 80 solution (1.5% a.q.) to 500 ml of a phosphate buffered solution, under conditions of 37° C. and 100 rpm. During the remaining three hours, the dissolution tests were performed at 37° C., 100 rpm in 500 ml of a phosphate buffered solution (pH 7.2). The results of the dissolution tests are shown in Table 4 below and FIG. 2.

TABLE 4

| | Released amount of | | |
|---|---|---|---|
| | tamsulosin hydrochloride (wt %) | | |
| Time (hrs) | Example 3 | Example 4 | Example 5 |
| 0 | 0 | 0 | 0 |
| 2 | 25.32 | 28.56 | 22.34 |
| 3 | 74.89 | 97.69 | 60.23 |
| 5 | 99.785 | 100.43 | 101.45 |

As seen from Table 4 and FIG. 2, 0.05 mg corresponding to about 25% of 0.2 mg of drugs contained in the tablets was gradually released for initial two hours and the balance (0.15 mg) was gradually released for the remaining three hours.

### EXAMPLES 6 AND 7

Evaluation of Acting Effect of Outer Coating Layer

In Example 6, tablets were prepared using the same composition and method as in Example 5 except that the coating amount of outer coating layers was 1% of the total weight of sustained-release cores.

In Example 7, tablets were prepared using the same composition and method as in Example 5 except that the coating amount of outer coating layers was 3% of the total weight of sustained-release cores.

Dissolution Test

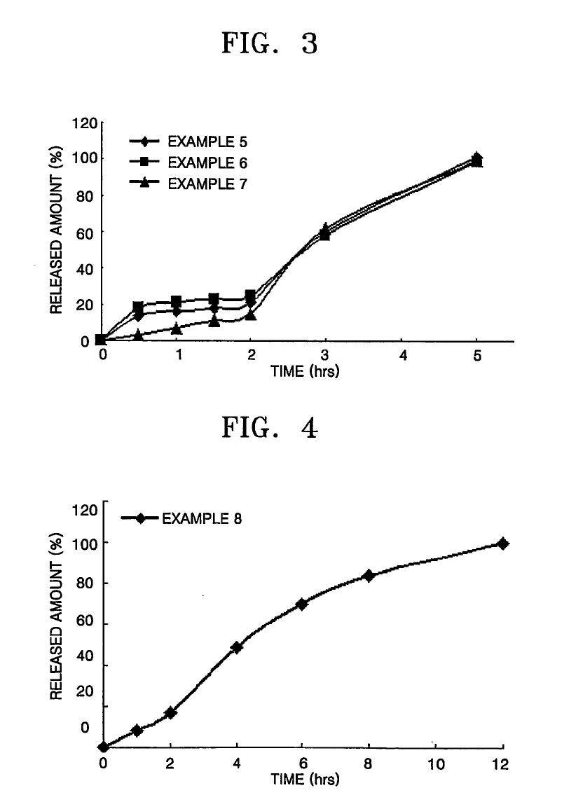

The dissolution tests for the above-prepared tablets were performed in the same manner as those for Examples 1 through 3. The results of the dissolution tests are shown in Table 5 below and FIG. 3.

TABLE 5

| | Released amount of | | |
|---|---|---|---|
| | tamsulosin hydrochloride (wt %) | | |
| Time (hrs) | Example 5 | Example 6 | Example 7 |
| 0 | 0 | 0 | 0 |
| 0.5 | 13.5 | 18 | 3 |
| 1 | 16 | 21 | 7 |
| 1.5 | 18 | 23 | 11 |
| 2 | 22.34 | 25 | 15 |
| 3 | 60.23 | 58 | 62 |
| 5 | 101.45 | 99 | 99.5 |

As seen from Table 5 and FIG. 3, about 0.05 mg of 0.2 mg drugs contained in the tablets was gradually released for initial two hours. The release rates varied according to the coating amount of the outer coating layer.

### EXAMPLE 8

Preparation of Tablets

Tablets of this Example had a composition as presented in Table 6 below.

TABLE 6

| Section | Weight (mg) |
|---|---|
| Pseudoephedrine hydrochloride | 120 |
| Cetirizine hydrochloride | 5 |
| Hipromelose | 99 |
| HPC | 30 |
| Magnesium stearate | 1 |
| HPMC 2910 | 14.75 |
| Talc | 0.45 |
| Triacetin | 1.5 |
| Tablet | 271.7 |

(1) Preparation of Sustained-release Cores

120 kg of pseudoephedrine hydrochloride, 99 kg of hydroxypropylmethylcellulose (HPMC) (hipromelos 2903 mPa·s), and 30.0 kg of a binder (hydroxypropylcellulose, HPC) were mixed in a mixer. Magnesium stearate (500 g) was added thereto and completely mixed. Ribbon-type granules were made using a roller compacter and screened through a 20-mesh screen. Magnesium stearate (500 g) was again added and mixed. The resultant mixture was compacted in a rotary press (Korsch PH 106) to make 100,000 white tablets (250.0 mg for each).

(2) Enteric Film Coating

10% coating was performed on the white tablets in the same manner as in Examples 3 through 7.

(3) Preparation and Coating of Drug Coating Suspension

HPMC 2910 mPa·s (14.75 kg), talc (0.45 kg), and triacetin (1.5 kg) were gradually added to water (35.007 kg), which had been heated to about 70° C., with stirring, and water (17.503 kg) was added thereto. Then, cetirizine hydrochloride (5 kg) was gradually added to prepare a drug coating suspension. The drug coating suspension was homogenized and cooled to room temperature with continuously stirring. The enteric film-coated tablets prepared in Section (2) were placed in a coating pan and warmed by air so that an outlet air temperature reached about 40 to 50° C. The drug coating suspension was sprayed on the enteric film-coated tablets. The resultant tablets with drug-containing film coating layers were dried by air supply for about 10 minutes, isolated from the coating pan, and stored.

By doing so, there were made tablets having an average weight of 271.7 mg and each containing 120 mg of pseudoephedrine and 5 mg of cetirizine hydrochloride.

(4) Dissolution Test

The dissolution test for the tablets prepared in Example 8 was performed according to the second method of the dissolution test in the Korean pharmacopoeia. At this time, the dissolution test was carried out under conditions of 37° C. and 100 rpm for 12 hours. The result of the dissolution test is presented in Table 7 below.

TABLE 7

Example 8

| Time | Pseudoephedrine | Cetirizine |
|---|---|---|
| (hrs) | hydrochloride (%) | hydrochloride (%) |
| 0 | 0 | 0 |
| 1 | 8.3 | 100 |
| 2 | 17 | — |
| 4 | 49 | — |
| 6 | 70 | — |
| 8 | 84 | — |
| 12 | 100 | — |

The released amount of pseudoephedrine hydrochloride, which was a drug contained in the sustained-release cores of the above-prepared tablets, was 10-20% for 1-2 hours and the released amount of cetirizine hydrochloride contained in active ingredient-containing film coating layers was 100% for 1-2 hours. The balance (90-80%) of the pesudoephedrine hydrochloride was gradually released for the remaining 10-11 hours.

1. A sustained-release formulation comprising:
(a) a sustained-release core comprising an active ingredient and a polymer having erosion and swelling property in mammalian intestinal secretions;
(b) an enteric film coating layer coated on the sustained-release core; and
(c) an active ingredient-containing film coating layer coated on the enteric film coating layer and comprising the active ingredient and a hydrophilic polymer for film coating.

2. The sustained-release formulation of claim 1, which further comprises an outer coating layer coated on the active ingredient-containing film coating layer and comprising a film coating polymer selected from the group consisting of a hydrophilic polymer, a hydrophobic polymer, a pH-dependent polymer, and a combination thereof.

3. The sustained-release formulation of claim 1 or 2, wherein the polymer contained in the sustained-release core is a polymer having a viscosity of 1 to 100,000 mPa·s.

4. The sustained-release formulation of claim 1 or 2, wherein the hydrophilic polymer contained in the active ingredient-containing film coating layer is a hydrophilic polymer having a viscosity of 1 to 100,000 mPa·s.

5. The sustained-release formulation of claim 1 or 2, wherein the polymer contained in the sustained-release core is selected from the group consisting of hydroxypropylmethylcellulose, hydroxypropylcellulose, hydroxyethylcellulose, hydroxymethylcellulose, polyethylene oxide, waxes (Carnauba wax), sodium alginate, povidone, polyvinylalcohol, sodium carboxymethylcellulose, xanthan gum, alginic acid salt and its derivative, and a combination thereof.

6. The sustained-release formulation of claim 5, wherein the polymer contained in the sustained-release core is hydroxypropylmethylcellulose.

7. The sustained-release formulation of claim 1 or 2, wherein the content of the polymer in the sustained-release core is 1 to 99 wt %, based on the total weight of the sustained-release core.

8. The sustained-release formulation of claim 1, wherein the enteric film coating layer includes an enteric polymer which is soluble at about pH 5 or more.

9. The sustained-release formulation of claim 2, wherein the polymer contained in the outer coating layer is an enteric polymer which is soluble at about pH 5 or more.

10. The sustained-release formulation of claim 8 or 9, wherein the enteric polymer is selected from the group consisting of cellulosic polymers, polyvinyl polymers, maleic acid vinyl polymers, polymethacrylate copolymers, and combinations thereof.

11. The sustained-release formulation of claim 8 or 9, wherein the enteric copolymer is a 1:1 copolymer of methacrylic acid and ethylacrylate.

12. The sustained-release formulation of claim 1 or 2, wherein the hydrophilic polymer contained in the active ingredient-containing film coating layer is selected from the group consisting of polyvinylalcohol, polyethyleneglycol, polypropyleneglycol, acrylic acid copolymer, hydroxypropylmethylcellulose, hydroxypropylcellulose, methylcellulose, ethylcellulose, and a combination thereof.

13. The sustained-release formulation of claim 1 or 2, wherein the active ingredient is a drug selected from the group consisting of antihypertensive agents, antidiabetes agents, antilipemic agents, cardiovascular drugs, expectrorants, antibiotics, emollients, steroids, antiasthmatic drugs, nonsteroid anti-inflammatory agents, therapeutic agents for prostatic enlargement, antidepressants, antihistamines, and combinations thereof.

14. The sustained-release formulation of claim 13, wherein the active ingredient is nifedipine, felodipine, cetirizine, pseudoephedrine, tamsulosin, or a pharmaceutically acceptable salt thereof.

15. The sustained-release formulation of claim 14, wherein the active ingredient is tamsulosin or its hydrochloride.

16. The sustained-release formulation of claim 15, wherein 60 to 99 wt % of tamsulosin is contained in the sustained-release core and 1 to 40 wt % of tamsulosin is contained in the active ingredient-containing film coating layer.
